(19) United States
(12) Patent Application Publication
(10) Pub. No.: US 2021/0213684 A1
(43) Pub. Date: Jul. 15, 2021

(54) CONSTRUCTION METHOD FOR CUSTOMIZATION OF MODULAR BONE PLATES BASED ON ADDITIVE MANUFACTURING AND A CONSTRUCTION SYSTEM THEREOF

(71) Applicants: Chih-Yu CHEN, Kaohsiung City (TW); INGROWTH BIOTECH CO., LTD., Kaohsiung City (TW)

(72) Inventors: Chih-Yu CHEN, Kaohsiung City (TW); Po-Han CHU, Kaohsiung City (TW)

(21) Appl. No.: 16/737,903

(22) Filed: Jan. 9, 2020

(51) Int. Cl. B29C 64/386; B33Y 50/00; A61B 17/80

(52) U.S. Cl. CPC B29C 64/386; A61B 2017/00526; A61B 17/80; B33Y 50/00

(57) ABSTRACT

A construction method for customization of modular bone plates based on additive manufacturing relies on a modularization technique to construct and save a plurality of bone plate modules in a database connected with an analysis unit through which collected images for patients' fractures or osteomiosis are simulated for recognizing an injury part and extent and estimating and displaying bone plates required on a specific area and/or extent. Some analyzed results of the analysis unit are read from an operation interface on which a user determines and selects the bone plate modules for a surgery operation; the files for the bone plate modules are transmitted to a remote manufacturing center through a processing unit and to an additive manufacturing apparatus from the manufacturing center for manufacture and output of finished bone plates with curved surfaces according to an additive manufacturing technology.

### BACKGROUND OF THE INVENTION

### 1. Field of the Invention

The present disclosure relates to a construction method for customization of modular bone plates based on additive manufacturing with which modularized medical bone plates are constructed and a construction system for customization of modular bone plates based on additive manufacturing through which the construction method is practically implemented.

### 2. Description of Related Art

Bone plates (also known as steel sheets) are common medical devices in treatment to fractures or osteomiosis. In the existing practice, a patient's injury part should be scanned in a hospital according to computed tomography (CT) through which CT images are produced; then, a technician has to create a separate skeleton module for the patient and prepare off-the-shelf bone plates with respect to the skeleton module for a surgery operation. The same procedure applicable to each patient without exception is time-consuming during the early stage to create the skeleton module.

However, for a specific bone position (for example, proximal or distal humerus, distal femur, proximal or distal tibia, or pelvis), a single or a few off-the-shelf bone plates with an identical shape but a longer or shorter length only are attainable.

Particularly, the length and/or the position of a bone plate to be used and each screw's length to be measured during hole-drilling are subject to a surgeon's decision in a surgery operation. Accordingly, a standard bone plate (which is a standardized 2-dimensional part not completely matching the shape of a dissected part at a patient and folded for better flexibility as required) should be bent during a surgery operation. However, bending a bone plate takes long time during which a bone plate hardly fits a bone position once.

Moreover, standardized bone plates possibly shorter in length and combined with each other are characteristic of inconsistent mechanical strengths. In particularly, a surgery operation for two bone plates mounted on a limited position is more difficult than expected.

Because a human skeleton is modified within a reasonable extent, a great number of bone plate modules should be created for a surgeon who can select appropriate bone plate modules for a patient directly without excessive time consumed during the early stage of creating the skeleton module. In the present disclosure, a construction method for customization of modular bone plates based on additive manufacturing and a construction system thereof are a solution with an additive manufacturing technology integrated for fast customized manufacturing of bone plates as required.

A construction method for customization of modular bone plates based on additive manufacturing and a construction system thereof in the present disclosure have the potential to develop a “Build to Oder (BTO)” model that features a design easily modified on a computer interface before manufacturing for no need to stock up spared bond plates in a traditional model.

### SUMMARY OF THE INVENTION

A construction method for customization of modular bone plates based on additive manufacturing in the present disclosure for treatment to fractures or osteomiosis by bone plates comprises steps as follows: step A: a plurality of bone plate modules are separated and simulated in software based on positions, areas and frequency of uses for off-the-shelf bone plates mounted on the human skeleton such that a great quantity of bone plate modules referring to various categorized body types are classified and each of the bone plate modules is saved as a transmittable and readable file one by one; step B: a file for each of the bone plate modules is categorized and saved in a database in which files about the human skeleton for various body types matched by the bone plate modules and files about screws with different sizes fitting the bone plate modules are saved simultaneously; step C: medical images for patients' fractures or osteomiosis are collected for simulations of medical images in an analysis unit connected with the database and files for the human skeleton, which are most approximate to the medical images and extracted from the database, as well as the bone plate modules corresponding to the human skeleton are compared and analyzed in the analysis unit with which an injury part and extent is recognized through comparisons and bone plates for the injury part and extent are further estimated and displayed to a user who will evaluate a surgery operation by severity of injury through the medical images and determine bone plates for the injury part and extent as well as screw with different sizes for the bone plate modules; step D: a combination of the bone plate modules and the screws with different sizes corresponding to the bone plate modules for a surgery operation are determined by a user who has read analyzed results of the analysis unit on an operation interface, selected through the operation interface, and transmitted to a remote manufacturing center by a processing unit;

Specifically, the medical images for a patient's fractures or osteomiosis are CT, MRI or X-ray images.

Specifically, the analysis unit in step C, which is used to identify an injury part and extent by checking and comparing medical images of a patient's fractures or osteomiosis, design and preset a group of bone plate modules according to severity of fractures automatically through introduction of intelligence analysis, estimate screws with different sizes for the bone plate modules early, and collect all data selected on the operation interface for integrated analysis, contributes to more accurate bone plate modules preset automatically than expected.

Specifically, the analysis unit in step C provides a surgeon with analyzed results which consist of synthesized actual medical images, intelligible reorganized medical images, or files for the human skeleton extracted from the database, all of which are freely selected by a user.

Specifically, either a single bone plate module or a combination of at least two optional bone plate modules for large-area coverage is available in step D.

Specifically, all bone plate modules, files about the human skeleton for various body types, and screws with different sizes to match the bone plate modules in step B are numbered for easy recognition and check in following steps including step D and step E.

Specifically, files for all selected bone plate modules and screws with different sizes, both of which have been received by the manufacturing center, are transmitted to an additive manufacturing apparatus for production and output of finished bone plates with curved surfaces on a basis of an additive manufacturing technology in step E after step D such that both the bone plates and prepared screws are provided to a user.

A construction system for customization of modular bone plates based on additive manufacturing in practice of the construction method for customization of modular bone plates based on additive manufacturing comprises at least a configuration platform and a manufacturing center. The configuration platform is provided with a import data unit, a database, an analysis unit, a processing unit and an operation interface wherein: the import data unit is used to read a medical image; the database is used to save a plurality of files for bone plate modules, a plurality of files about the human skeleton for various body types matched by the bone plate modules, and a plurality of files about screws with different sizes fitting the bone plate modules; the analysis unit which is connected with the import data unit, the database, the processing unit and the operation interface is used to select data in the database and compare the data with medical images in the import data unit for analysis of an injury part and display of analyzed results on the operation interface from which all bone plate modules and corresponding screws for a surgery operation are selected by a user and sent to the processing unit. The manufacturing center connected with the processing unit is interiorly provided with an additive manufacturing apparatus with which each bone plate module is output based on an additive manufacturing technology and practically manufactured as a finished bone plate with a curved surface.

In a preferred embodiment, the analysis unit is interiorly provided with an intelligence analysis module through which a group of bone plate modules based on analyzed results of the analysis unit for status of fractures are preset automatically as required.

In a preferred embodiment, the intelligence analysis module is used to estimate screws with different sizes for the bone plate modules.

In a preferred embodiment, the medical images are CT, MRI or X-ray images.

### BRIEF DESCRIPTION OF THE DRAWINGS

FIG. 1 is a schematic view which illustrates analyzed results of a construction method for customization of modular bone plates based on additive manufacturing in step C.

FIG. 2 is a schematic view which illustrates optional items on an operation interface of a construction method for customization of modular bone plates based on additive manufacturing in step D.

FIG. 3 is a schematic view which illustrates finished bone plates in a production & shipment step according to a construction method for customization of modular bone plates based on additive manufacturing.

FIG. 4 is a block diagram for configuration of system architecture in a construction system for customization of modular bone plates based on additive manufacturing.

### DETAILED DESCRIPTION OF THE PREFERRED EMBODIMENTS

The technical details, features and effects of a construction method for customization of modular bone plates based on additive manufacturing and a system thereof are clearly presented in preferred embodiments and accompanying drawings herein.

In the present disclosure, a construction method for customization of modular bone plates based on additive manufacturing in which a bone plate is prepared for treatment to fractures or osteomiosis comprises steps as follows:

Step A: a plurality of bone plate modules are separated and simulated in software based on positions, areas, frequency of uses and service efficiency for off-the-shelf bone plates mounted on the human skeleton such that a great quantity of bone plate modules referring to various categorized body types are classified and each of the bone plate modules is saved as a transmittable and readable file one by one;

Step B: a file for each of the bone plate modules is categorized and saved in a database in which files about the human skeleton for various body types matched by the bone plate modules and files about screws with different sizes fitting the bone plate modules are saved simultaneously;

Step C: medical images (for example, CT, MRI or X-ray images) for patients' fractures or osteomiosis are collected for simulations of medical images in an analysis unit connected with the database; files for the human skeleton, which are most approximate to the medical images and extracted from the database, and the bone plate modules corresponding to the human skeleton are compared and analyzed in the analysis unit with which an injury part and extent is recognized through comparisons and bone plates for the injury part and extent are further estimated and partially displayed to a surgeon who will evaluate a surgery operation by severity of injury through the medical images and determine bone plates for the injury part and extent as well as screw with different sizes for the bone plate modules;

Step D: a combination of the bone plate modules and the screws with different sizes corresponding to the bone plate modules for a surgery operation are determined by a surgeon who has read analyzed results of the analysis unit on an operation interface, selected through the operation interface, and transmitted to a remote manufacturing center by a processing unit; and

Step E: files for all bone plate modules and screws with different sizes, both of which have been received by the manufacturing center, are transmitted to an additive manufacturing apparatus from which bone plate products with curved surfaces are produced and the finished bone plates as well as prepared screws with different sizes are delivered to a surgeon who is ready to perform a surgery operation.

As shown below, a hypothetical example is presented for explanations and practices of the above steps:

A: A patient in an emergency case is first diagnosed with suspected osteomiosis;

B: The routine checks including computed tomography (CT) , Magnetic Resonance Imaging (MRI) or X-ray are conducted and scanned medical images are transmitted to an analysis unit and a surgeon for step C;

C: A surgery operation is evaluated by the surgeon who inspects severity of injury from medical images and determines bone plates for an injury part and extent as well as screws with different sizes for the bone plate modules;

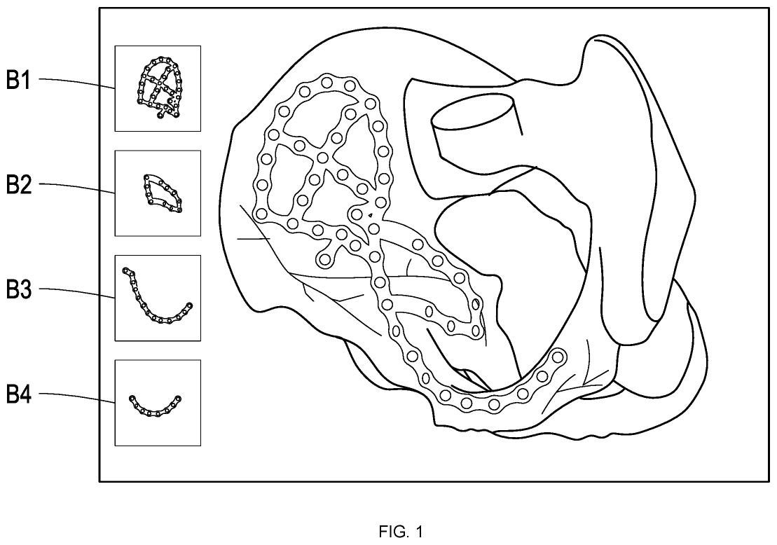

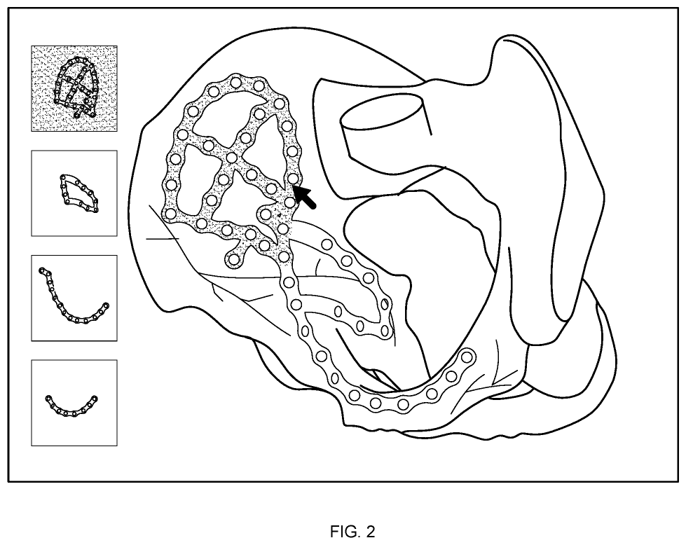

D: An operation interface from which the analysis unit is accessed controllably and results in stage C are displayed is available to the surgeon, as shown in FIG. 1, and a combination of the bone plate modules as well as screws with different sizes are configured and/or selected by the surgeon, as shown in FIG. 2;

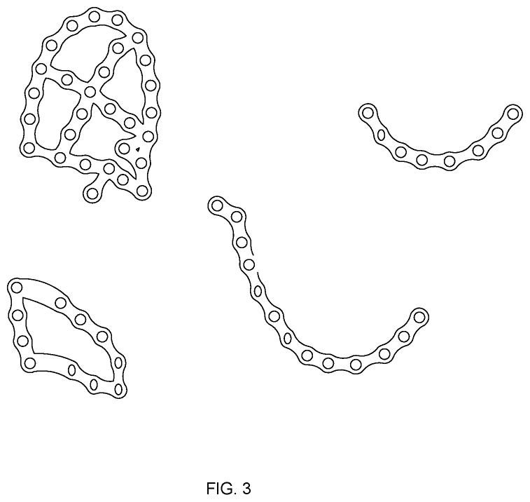

E: The combination of bone plate modules as well as screws, configured and selected in stage D (FIG. 3), are output for preparation of bone plate modules at a remote manufacturing center from which a combination of bone plate products with curved surfaces produced on a basis of an additive manufacturing technology and corresponding screws are transmitted to the surgeon quickly; and

F: A surgery operation is performed forthwith.

In a construction method for customization of modular bone plates based on additive manufacturing in the present disclosure, the analysis unit in step C is used to identify an injury part and extent by checking and comparing medical images of a patient's fractures or osteomiosis, design and preset a group of bone plate modules according to severity of fractures automatically through introduction of intelligence analysis, and estimate screws with different sizes for the bone plate modules early. Accordingly, both a combination of bone plate modules and screws with different sizes are quickly selected by a surgeon who has reviewed analyzed results of the analysis unit on the operation interface.

Because all data for bone plate modules and screws are selected on the operation interface for integrated analysis by the analysis unit, the accuracy of the bone plate modules preset automatically will be promoted gradually for easy operation of the medical staff or ordinary operators in addition to surgeons in days to come.

In a construction method for customization of modular bone plates based on additive manufacturing in the present disclosure, the analysis unit in step C provides a surgeon with analyzed results which consist of synthesized actual medical images, intelligible reorganized medical images, or files for the human skeleton extracted from the database, all of which are freely selected by the surgeon.

In a construction method for customization of modular bone plates based on additive manufacturing in the present disclosure, the bone plate module for a pelvis in an embodiment as shown in FIG. 1 can be chosen from four types, B1, B2, B3 and B4, presumptively. Based on a request for a surgery operation, either a single bone plate module or a combination of different modules for large-area coverage selected from types B1, B2, B3 and B4 is available to a surgeon in step D. For customization, this concept is applicable to all bone positions of the whole human body.

In a construction method for customization of modular bone plates based on additive manufacturing in the present disclosure, all bone plate modules, files about the human skeleton for various body types, and screws with different sizes to match the bone plate modules in step B are numbered for easy recognition and check in following steps including step D and step E.

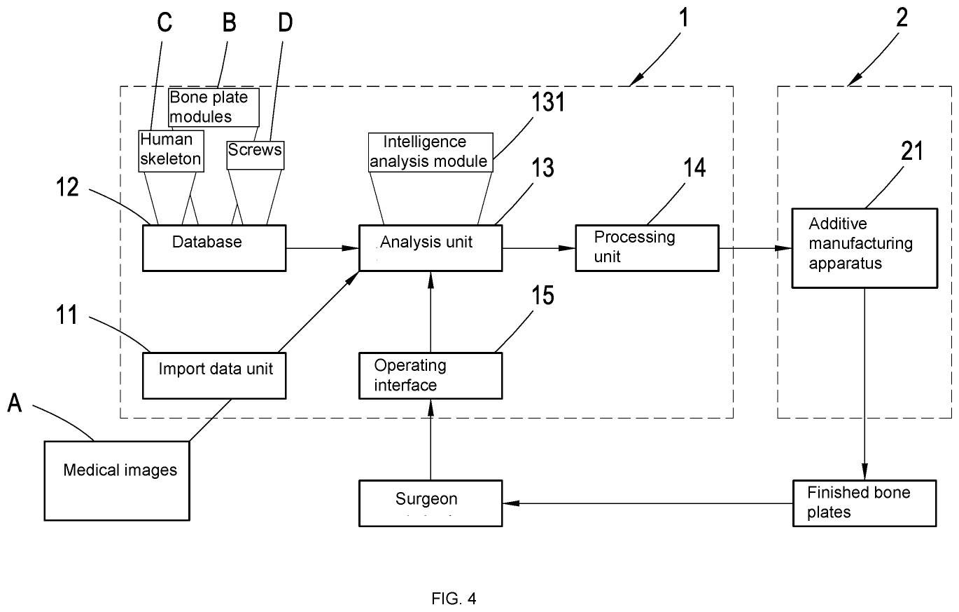

Referring to FIG. 4, which is a block diagram for configuration of system architecture and illustrates a construction system for customization of modular bone plates based on additive manufacturing in practice of the construction method for customization of modular bone plates based on additive manufacturing comprises at least a configuration platform 1 and a manufacturing center 2.

In the construction system for customization of modular bone plates based on additive manufacturing, the configuration platform 1 comprises a import data unit 11, a database 12, an analysis unit 13, a processing unit 14 and an operation interface 15: the import data unit 11 is used to read a medical image A (a CT, MRI or X-ray image in general); the database 12 is used to save a plurality of files for bone plate modules B, a plurality of files about the human skeleton C for various body types matched by the bone plate modules B, and a plurality of files about screws D with different sizes fitting the bone plate modules B; the analysis unit 13 which is connected with the import data unit 11, the database 12, the processing unit 14 and the operation interface 15 is used to select data in the database 12 and compare the data with images in the import data unit 11 for analysis of an injury part and display of analyzed results on the operation interface 15 from which all bone plate modules B and corresponding screws D for a surgery operation are selected by an operator and sent to the processing unit 14.

In the construction system for customization of modular bone plates based on additive manufacturing, the manufacturing center 2 connected with the processing unit 14 is used to receive instructions from the processing unit 14 for preparation of medical devices for a surgery and provided with an additive manufacturing apparatus with which each bone plate module B is output on the basis of an additive manufacturing technology and practically manufactured as a finished bone plate with a curved surface.

Referring to FIG. 4 that illustrates the construction system for customization of modular bone plates based on additive manufacturing wherein the analysis unit 13 is interiorly provided with an intelligence analysis module 131 through which a group of bone plate modules B based on analyzed results for status of fractures are preset automatically and screws D with different sizes for the bone plate modules B are estimated.

Compared with prior arts, a construction method for customization of modular bone plates based on additive manufacturing and a construction system thereof provided in the present disclosure features advantages as follows:
(1) According to the concept of modularization, a construction method for customization of modular bone plates based on additive manufacturing and a construction system thereof through which the accuracy of bone plate modules is promoted due to integrated analysis of each data in step C are easily operated by ordinary medical staff or technicians in addition to surgeons for creating a combination of required bone plate modules quickly.
(2) Based on the concept of modularization, customized bone plate modules are fast combined and flexibly manufactured for the time-saving front-end process of computer graphics for bone plates, reduced total manufacturing time and promotion of medical quality and efficiency.
(3) As shown in step D, the analyzed results of the analysis unit displayed to a surgeon consist of synthesized actual medical images, intelligible reorganized medical images, or files for the human skeleton extracted from the database, all of which are freely selected by a surgeon who can easily adjust a surgery operation program and a position of a bone plate to be mounted for effective modifications of bone plate modules and designs of required bone plate modules without extra cost.
(4) Additive manufacturing contributes to fast customization of bone plate modules to be mounted at all positions of a human body as required and real-time selection and manufacture of bone plate modules for almost no spare part, reduced inventory cost and even remote manufacturing.
(5) Additive manufacturing is applicable to special cases; for example, made-to-order bone plates for treatment to rare fractures or ultra-long bone plates for treatment to ultra-long bone fractures are fast planned and manufactured through additive manufacturing or modularization.
(6) A bone plate module produced with additive manufacturing can be post-processed in a standard procedure for promotion of mechanical strength and conformity with a standard manufacturing process of the medical equipment inspection and registration.
(7) Finished bone plates produced with additive manufacturing satisfy an individual patient's anatomical structure for no repeated adjustment of the curvature of a bone plate, time saving for a surgery operation, and reference and prediction to the length of a screw without any complication due to a screw longer or shorter.

The preferred embodiments hereof are not taken as examples to restrict the scope of a construction method for customization of modular bone plates based on additive manufacturing and a construction system thereof in the present disclosure. Any equivalent change and/or modification made by the skilled persons who familiarize themselves with the above technical features and embodiments without departing from the spirit and scope of the present disclosure should be covered in claims of the patent specification.

What is claimed is:

1. A construction method for customization of modular bone plates based on additive manufacturing used in treatment to fractures or osteomiosis by bone plates and comprising steps as follows:
step A: a plurality of bone plate modules are separated and simulated in software based on positions, areas and frequency of uses for off-the-shelf bone plates mounted on the human skeleton such that a great quantity of bone plate modules referring to various categorized body types are classified and each of the bone plate modules is saved as a transmittable and readable file one by one;
step B: a file for each of the bone plate modules is categorized and saved in a database in which files about the human skeleton for various body types matched by the bone plate modules and files about screws with different sizes fitting the bone plate modules are saved simultaneously;
step C: medical images for patients' fractures or osteomiosis are collected for simulations of medical images in an analysis unit connected with the database and files for the human skeleton, which are most approximate to the medical images and extracted from the database, as well as the bone plate modules corresponding to the human skeleton are compared and analyzed in the analysis unit with which an injury part and extent is recognized through comparisons and bone plates for the injury part and extent are further estimated and displayed to a user who will evaluate a surgery operation by severity of injury through the medical images and determine bone plates for the injury part and extent as well as screw with different sizes for the bone plate modules;
step D: a combination of the bone plate modules and the screws with different sizes corresponding to the bone plate modules for a surgery operation are determined by a user who has read analyzed results of the analysis unit on an operation interface, selected through the operation interface, and transmitted to a remote manufacturing center by a processing unit.

2. The construction method for customization of modular bone plates based on additive manufacturing as claimed in claim 1 wherein the medical images for a patient's fractures or osteomiosis are CT, MRI or X-ray images.

3. The construction method for customization of modular bone plates based on additive manufacturing as claimed in claim 1 wherein the analysis unit in step C, which is used to identify an injury part and extent by checking and comparing medical images of a patient's fractures or osteomiosis, design and preset a group of bone plate modules according to severity of fractures automatically through introduction of intelligence analysis, estimate screws with different sizes for the bone plate modules early, and collect all data selected on the operation interface for integrated analysis, contributes to more accurate bone plate modules preset automatically than expected.

4. The construction method for customization of modular bone plates based on additive manufacturing as claimed in claim 1 wherein the analysis unit in step C provides a surgeon with analyzed results which consist of synthesized actual medical images, intelligible reorganized medical images, or files for the human skeleton extracted from the database and freely selected by a user.

5. The construction method for customization of modular bone plates based on additive manufacturing as claimed in claim 1 wherein either a single bone plate module or a combination of at least two optional bone plate modules for large-area coverage is available in step D.

6. The construction method for customization of modular bone plates based on additive manufacturing as claimed in claim 1 wherein all bone plate modules, files about the human skeleton for various body types, and screws with different sizes to match the bone plate modules in step B are numbered for easy recognition and check in following steps including step D and step E.

7. The construction method for customization of modular bone plates based on additive manufacturing as claimed in claim 1 wherein files for all selected bone plate modules and screws with different sizes, both of which have been received by the manufacturing center, are transmitted to an additive manufacturing apparatus for production and output of finished bone plates with curved surfaces on a basis of an additive manufacturing technology in step E after step D such that both the bone plates and prepared screws are provided to a user.

8. A construction system for customization of modular bone plates based on additive manufacturing, comprising at least:
a configuration platform provided with a import data unit, a database, an analysis unit, a processing unit and an operation interface wherein: the import data unit is used to read a medical image; the database is used to save a plurality of files for bone plate modules, a plurality of files about the human skeleton for various body types matched by the bone plate modules, and a plurality of files for screws with different sizes fitting the bone plate modules; the analysis unit connected with the import data unit, the database, the processing unit and the operation interface is used to select data in the database and compare the data with medical images in the import data unit for analysis of an injury part and display of analyzed results on the operation interface from which all bone plate modules and corresponding screws for a surgery operation are selected by a user and sent to the processing unit;
a manufacturing center connected with the processing unit is interiorly provided with an apparatus with which each bone plate module is output and practically manufactured as a finished bone plate with a curved surface.

9. The construction system for customization of modular bone plates based on additive manufacturing as claimed in claim 8 wherein the analysis unit is interiorly provided with an intelligence analysis module through which a group of bone plate modules on the basis of analyzed results of the analysis unit for status of fractures are preset automatically as required.

10. The construction system for customization of modular bone plates based on additive manufacturing as claimed in claim 9 wherein the intelligence analysis module is used to estimate screws with different sizes for the bone plate modules.

11. The construction system for customization of modular bone plates based on additive manufacturing as claimed in claim 8 wherein the medical images are CT, MRI or X-ray images.

12. The construction system for customization of modular bone plates based on additive manufacturing as claimed in claim 8 wherein the apparatus is an additive manufacturing apparatus.
